(19) United States
(12) Patent Application Publication
(10) Pub. No.: US 2025/0108204 A1
(43) Pub. Date: Apr. 3, 2025

(54) Delivering Tumor Treating Fields (TTFields) to a Subject's Brain Using Electrodes Installed in Through-Holes in the Subject's Skull

(71) Applicant: Novocure GmbH, Root D4 (CH)

(72) Inventor: Steven A. TOMS, Providence, RI (US)

(73) Assignee: Novocure GmbH, Root D4 (CH)

(21) Appl. No.: 18/897,192

(22) Filed: Sep. 26, 2024

Related U.S. Application Data
(60) Provisional application No. 63/541,344, filed on Sep. 29, 2023.

(51) Int. Cl. A61N 1/05; A61N 1/36

(52) U.S. Cl. CPC A61N 1/0529; A61N 1/36002

(57) ABSTRACT

When Tumor Treating Fields (TTFields) are applied to a subject's brain through the subject's skull, the skull dramatically attenuates the amplitude of the TTFields. The attenuating effect of the skull can be surmounted by making small through-holes in the skull, and installing individual electrodes in each of those through-holes so that the inner ends of those individual electrodes are positioned beneath the subject's skull. Then, when an AC voltage is applied between the electrodes on one side of the subject skull and the electrodes on the opposite side of the subject's skull, the resulting electric field will not pass through the skull and will therefore not be attenuated.

### CROSS REFERENCE TO RELATED APPLICATIONS

This application claims the benefit of U.S. Provisional Application 63/541,344, filed Sep. 29, 2023, which is incorporated herein by reference in its entirety.

### BACKGROUND

Tumor Treating Fields (TTFields) therapy is a proven approach for treating brain tumors (e.g., glioblastoma) using alternating electric fields at 100-500 kHz (e.g., 200 kHz). In the prior art Optune® system, TTFields are delivered to patients via four transducer arrays that are placed on the patient's skin near the tumor. The transducer arrays are arranged in two pairs, with one pair of transducer arrays positioned to the left and right of the tumor, and the other pair of transducer arrays positioned anterior and posterior to the tumor. When an AC voltage is applied between opposing electrode assemblies, an AC current is coupled through the electrode assemblies and into the subject's body, which induces an electric field in the target region (e.g., the tumor). And higher field strengths are strongly correlated with higher efficacy of treatment.

Conventional electrode assemblies for applying TTFields to a subject's body are applied to the subject's skin on opposite sides of the head, and examples of conventional electrode assemblies are described in U.S. Pat. No. 8,715,203, and Pub. No. US 2021/0202179. But when the electrodes are positioned on the subject's skin, the electric field must pass through the patient's scalp and skull twice in order to reach the tumor, and this introduces two difficulties. First, the presence of the skull between the transducer array and the tumor makes it more difficult to aim the field at the desired location (i.e., the tumor bed) in the brain. And second, due to attenuation of the electric field introduced by the skull and scalp, the voltage and current that is applied to the transducer arrays must be relatively high in order to obtain an electric field with a therapeutically effective magnitude in the tumor bed.

### SUMMARY OF THE INVENTION

One aspect of the invention is directed to a first apparatus for delivering alternating electric fields to a target region in a subject's brain. The first apparatus comprises a plurality of first electrode assemblies configured for positioning through a respective plurality of first holes in the subject's skull on a first side of the target region, and a plurality of second electrode assemblies configured for positioning through a respective plurality of second holes in the subject's skull on a second side of the target region. Each of the plurality of first electrode assemblies has a first shaft having a longitudinal axis, an inner end, and an outer end, wherein the first shaft is shaped and dimensioned to traverse a respective one of the first holes; a first flange disposed at the outer end of the first shaft, wherein the first flange has a larger outer diameter than the first shaft and is shaped and dimensioned to prevent the first electrode assembly from passing through the respective first hole; a first conductive electrode element disposed at the inner end of the first shaft, wherein the first electrode element has an inner face that is within 10° of perpendicular to the longitudinal axis of the first shaft, and wherein the inner face of the first electrode element has an area of at least 5 mm²; and a first conductive wire having a first portion that is positioned in electrical contact with the first electrode element and a second portion that is configured to run beneath the subject's scalp. Each of the plurality of second electrode assemblies has a second shaft having a longitudinal axis, an inner end, and an outer end, wherein the second shaft is shaped and dimensioned to traverse a respective one of the second holes; a second flange disposed at the outer end of the second shaft, wherein the second flange has a larger outer diameter than the second shaft and is shaped and dimensioned to prevent the second electrode assembly from passing through the respective second hole; a second conductive electrode element disposed at the inner end of the second shaft, wherein the second electrode element has an inner face that is within 10° of perpendicular to the longitudinal axis of the second shaft, and wherein the inner face of the second electrode element has an area of at least 5 mm²; and a second conductive wire having a first portion that is positioned in electrical contact with the second electrode element and a second portion that is configured to run beneath the subject's scalp. The first apparatus also comprises at least one port configured for affixation to the subject's skull. The at least one port includes (a) at least one first terminal configured to make an electrical connection with each of the first conductive wires so that an electrical signal can be applied to each of the first conductive wires via the at least one port, and (b) at least one second terminal configured to make an electrical connection with each of the second conductive wires so that an electrical signal can be applied to each of the second conductive wires via the at least one port.

In some embodiments of the first apparatus, each of the plurality of first electrode assemblies has a first layer of insulating material disposed on the inner face of the first electrode element. The first layer of insulating material is positioned to insulate the first electrode element from the subject's dura when the first shaft is positioned in the respective first hole. And the first layer of insulating material has a dielectric constant of at least 10. In these embodiments, each of the plurality of second electrode assemblies has a second layer of insulating material disposed on the inner face of the second electrode element. The second layer of insulating material is positioned to insulate the second electrode element from the subject's dura when the second shaft is positioned in the respective second hole. And the second layer of insulating material has a dielectric constant of at least 10.

Optionally, in the embodiments described in the previous paragraph, each of the first layers of insulating material and each of the second layers of insulating material comprises a polymer layer having a thickness of less than 50 μm. Optionally, in the embodiments described in the previous paragraph, each of the first layers of insulating material and each of the second layers of insulating material comprises a ceramic material having a dielectric constant of at least 1000.

In some embodiments of the first apparatus, each of the first electrode elements is positioned to contact the subject's dura when the respective first shaft is positioned in the respective first hole, and each of the second electrode elements is positioned to contact the subject's dura when the respective second shaft is positioned in the respective second hole. Optionally, in these embodiments, each of the first electrode elements and each of the second electrode elements is made of metal.

In some embodiments of the first apparatus, the inner faces of each of the first electrode elements and each of the second electrode elements has an area of 5-20 mm². In some embodiments of the first apparatus, each of the first electrode elements and each of the second electrode elements has a lower face that is within 2° of perpendicular to the longitudinal axis of the respective shaft.

Another aspect of the invention is directed to a first method for delivering alternating electric fields to a target region in a subject's brain. The first method comprises positioning a plurality of first electrode assemblies through a respective plurality of first holes in the subject's skull on a first side of the target region, and positioning a plurality of second electrode assemblies through a respective plurality of second holes in the subject's skull on a second side of the target region. Each of the plurality of first electrode assemblies has a first shaft having a longitudinal axis, an inner end, and an outer end, wherein the first shaft is shaped and dimensioned to traverse a respective one of the first holes; a first flange disposed at the outer end of the first shaft, wherein the first flange has a larger outer diameter than the first shaft and is shaped and dimensioned to prevent the first electrode assembly from passing through the respective first hole; a first conductive electrode element disposed at the inner end of the first shaft, wherein the first electrode element has an inner face that is within 10° of perpendicular to the longitudinal axis of the first shaft, and wherein the inner face of the first electrode element has an area of at least 5 mm²; and a first conductive wire having a first portion that is positioned in electrical contact with the first electrode element and a second portion that is configured to run beneath the subject's scalp. And each of the plurality of second electrode assemblies has a second shaft having a longitudinal axis, an inner end, and an outer end, wherein the second shaft is shaped and dimensioned to traverse a respective one of the second holes; a second flange disposed at the outer end of the second shaft, wherein the second flange has a larger outer diameter than the second shaft and is shaped and dimensioned to prevent the second electrode assembly from passing through the respective second hole; a second conductive electrode element disposed at the inner end of the second shaft, wherein the second electrode element has an inner face that is within 10° of perpendicular to the longitudinal axis of the second shaft, and wherein the inner face of the second electrode element has an area of at least 5 mm²; and a second conductive wire having a first portion that is positioned in electrical contact with the second electrode element and a second portion that is configured to run beneath the subject's scalp. The first method also comprises applying an alternating voltage that has a frequency between 50 kHz and 1 MHz between (a) the plurality of first electrode assemblies and (b) the plurality of second electrode assemblies.

In some instances of the first method, each of the plurality of first electrode assemblies has a first layer of insulating material disposed on the inner face of the first electrode element, the first layer of insulating material is positioned to insulate the first electrode element from the subject's dura when the first shaft is positioned in the respective first hole, and the first layer of insulating material has a dielectric constant of at least 10. In these instances, each of the plurality of second electrode assemblies has a second layer of insulating material disposed on the inner face of the second electrode element, the second layer of insulating material is positioned to insulate the second electrode element from the subject's dura when the second shaft is positioned in the respective second hole, and the second layer of insulating material has a dielectric constant of at least 10.

Optionally, in the instances described in the previous paragraph, each of the first layers of insulating material and each of the second layers of insulating material comprises a polymer layer having a thickness of less than 50 μm. Optionally, in the instances described in the previous paragraph, each of the first layers of insulating material and each of the second layers of insulating material comprises a ceramic material having a dielectric constant of at least 1000.

In some instances of the first method, each of the first electrode elements is positioned to contact the subject's dura when the respective first shaft is positioned in the respective first hole, and each of the second electrode elements is positioned to contact the subject's dura when the respective second shaft is positioned in the respective second hole. Optionally, in these instances, each of the first electrode elements and each of the second electrode elements is made of metal.

In some instances of the first method, the inner faces of each of the first electrode elements and each of the second electrode elements has an area of 5-20 mm². In some instances of the first method, each of the first electrode elements and each of the second electrode elements has a lower face that is within 2° of perpendicular to the longitudinal axis of the respective shaft. In some instances of the first method, the alternating voltage has a frequency between 100 kHz and 300 kHz.

Another aspect of the invention is directed to a second apparatus for delivering alternating electric fields to a target region in a subject's brain. The second apparatus comprises a shaft having a longitudinal axis, an upper end, and a lower end, wherein the shaft has a length of 4-10 mm and an outer diameter of 2-15 mm; a flange disposed at the upper end of the shaft, wherein the flange has a diameter that is at least 2 mm larger than the outer diameter of the shaft; a conductive electrode element disposed at the lower end of the shaft, wherein the electrode element has a lower face that is within 10° of perpendicular to the longitudinal axis of the shaft, and wherein the lower face of the electrode element has an area of at least 5 mm²; and a layer of insulating material disposed on the lower face of the electrode element. The layer of insulating material covers the lower face of the electrode element so as to prevent the lower face of the electrode element from coming into contact with tissue positioned below the apparatus. And the layer of insulating material has a dielectric constant of at least 10.

Some embodiments of the second apparatus further comprise a metal wire having a first section that is disposed in electrical contact with the electrode element and runs through the shaft. In these embodiments, the electrode element is made of metal.

Optionally, in the embodiments described in the previous paragraph, the layer of insulating material comprises a polymer layer having a thickness of less than 50 μm. Optionally, in the embodiments described in the previous paragraph, the layer of insulating material comprises a ceramic material having a dielectric constant of at least 1000.

In some embodiments of the second apparatus, the lower face of the electrode element has an area of 5-20 mm². In some embodiments of the second apparatus, the shaft has a cylindrical outer surface. In some embodiments of the second apparatus, the lower face of the electrode element is within 2° of perpendicular to the longitudinal axis of the shaft.

### BRIEF DESCRIPTION OF THE DRAWINGS

FIG. 1 depicts a system that can be used to apply TTFields to a subject's brain without encountering the attenuating effects of the subject's skull.

FIG. 2 depicts one type of electrode assembly installed through a subject's skull.

FIG. 3 depicts another type of electrode assembly installed through a subject's skull.

Various embodiments are described in detail below with reference to the accompanying drawings, wherein like reference numerals represent like elements.

### DESCRIPTION OF THE PREFERRED EMBODIMENTS

U.S. Pat. No. 11,654,279 solves both of the difficulties identified above by incorporating the electrodes into the inside of a skull implant that is shaped and dimensioned to replace a portion of the person's skull. But while this solution overcomes the difficulties of the skull interfering with the electric field, it introduces a different set of problems: (1) it is a highly invasive approach because large portions of the subject skull must be sawed away from the person's head, and (2) it can leave the person's head vulnerable to impacts.

This application describes a different approach for overcoming the attenuating effect that the skull has on TTFields. More specifically, instead of overcoming the attenuating effect of the skull by replacing an entire section of the skull with a skull implant that includes a set of interior electrodes, the attenuating effect of the skull is overcome by making a large number of small, less invasive through holes in the skull, and installing a plurality of individual electrodes in each of those through holes. And notably, because the inner ends of those individual electrodes are positioned beneath the subject's skull, AC currents that are introduced using those electrodes will not be attenuated by the skull. Advantageously, it is easier and less risky to introduce a set of small holes through the skull (as compared to replacing an entire section of the skull with the skull implant). Moreover, a set of small holes maintains the original structural integrity of the skull much better than a skull implant.

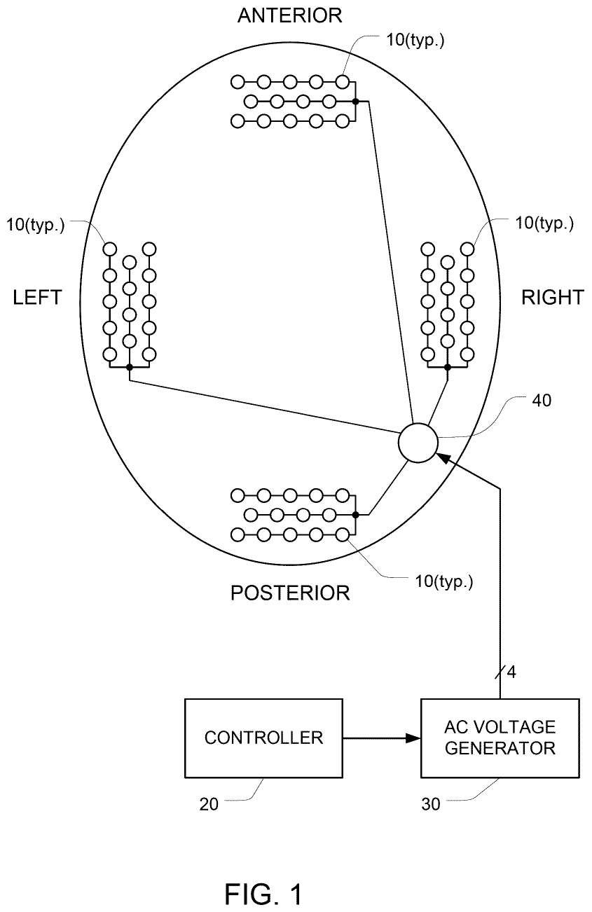

FIG. 1 depicts a system that can be used to apply TTFields to a subject's brain without encountering the attenuating effects of the subject's skull. To use this system, four sets of small holes are made on the left, right, anterior, and posterior sides of the subject's skull, and an individual electrode assembly 10 is installed in each of those holes. Note that while FIG. 1 shows 14 individual electrode assemblies 10 installed on each of the four sides of the subject's head, the number of individual electrode assemblies 10 on any of the four sides can vary (e.g., from 1-50, 2-25, 4-20, 4-15, etc.). In addition, while FIG. 1 shows four sets of electrode assemblies 10, the number of sets of electrode assemblies 10 can vary, e.g., from 2-10. When only two sets of electrode assemblies 10 are installed, they should be positioned on opposite sides of the subject's skull (e.g., one set on the left and one set on the right, or one set anterior and one set posterior).

After the sets of electrode assemblies 10 have been installed on the respective sides of the subject's head, an AC voltage generator 30 can apply AC current between opposing sets of electrodes (i.e., between the left and right sets of electrode assemblies, or between the anterior and posterior sets of electrode assemblies). The operation of the AC voltage generator 30 is controlled by a controller 20. A variety of approaches for implementing the AC voltage generator 30 can be used, including but not limited to the approaches described in U.S. Pat. No. 11,601,067, which is incorporated herein by reference in its entirety. In some embodiments, the AC voltage generator 30 and the controller 20 may be combined into a single device.

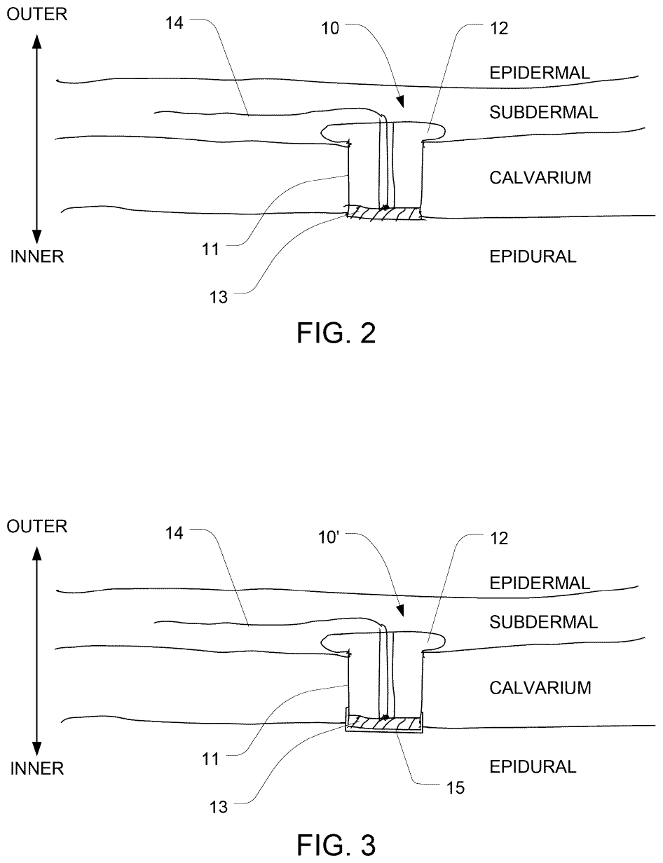

In some embodiments, the electrode assemblies 10 are installed into the anatomic location depicted in FIG. 2, so that the inner tip of the electrode element 13 lies in the epidural space in contact with the outer layer of the dura. Notably, the inner tip of the electrode assembly 10 does not go through the dura.

One suitable approach for installing the electrode assemblies 10 at this anatomic location is similar to the way that PEG epidural electrodes are installed. More specifically, each electrode assembly 10 can be installed into the anatomic location depicted in FIG. 2 by making a small (e.g., 1 cm) stab incision, drilling a pilot hole and inserting the electrode assembly 10 into the pilot hole, so that the inner tip of the electrode element 13 lies in the epidural space in contact with the outer layer of the dura. But a variety of alternative approaches can also be used to install the electrode assemblies 10 at the anatomical location depicted in FIG. 2.

In the embodiment depicted in FIG. 2, each of the electrode assemblies 10 has a shaft 11 that has a longitudinal axis, an inner end, and an outer end. The shaft is shaped and dimensioned to traverse a respective one of the holes in the skull. In some preferred embodiments, the shaft has a length of 4-10 mm and an outer diameter of 2-15 mm.

Each of the electrode assemblies 10 also has a flange 12 disposed at the outer end of the shaft, and the flange has a larger outer diameter than the shaft and is shaped and dimensioned to prevent the electrode assembly from passing through the respective hole. In some preferred embodiments, the flange 12 has a diameter that is at least 2 mm larger than the outer diameter of the shaft 11.

Each of the electrode assemblies 10 also has a conductive electrode element 13 disposed at the inner end of the shaft. The electrode element has an inner face that is within 10° of perpendicular to the longitudinal axis of the shaft (or within 2° in some preferred embodiments), and the inner face of the electrode element has an area of at least 5 mm². Each of the electrode assemblies 10 also has a conductive wire 14 that has a first portion that is positioned in electrical contact with the electrode element 13 and a second portion that is configured to run beneath the subject's scalp. In some preferred embodiments, the inner faces of each of the electrode elements 13 has an area of 5-20 mm².

In the FIG. 2 embodiment, each of the electrode elements 13 is positioned to conductively contact the subject's dura when the respective shaft is positioned in its respective hole through the skull. Each of the electrode elements 13 is made from a conductive material e.g., a biocompatible metal such as stainless steel. As a result, when the first set of electrode assemblies 10 is installed on one side of the subject's head and a second set of electrode assemblies 10 is installed on the opposite side of the subject's head, and an AC voltage is applied between the electrode elements 13 in those two sets of electrode assemblies 10 (via the wires 14), an AC current will be conductively coupled through the electrode assemblies 10 into the subject's brain.

The FIG. 3 embodiment is similar to the FIG. 2 embodiment, except that the FIG. 3 embodiment has an additional layer of insulating material 15 disposed on the inner face of the electrode element 13 (i.e., the lower face in FIG. 3). This layer of insulating material 15 is positioned to insulate the electrode element 13 from the subject's dura when the shaft is positioned in the respective hole through the skull. As a result, when the first set of electrode assemblies 10′ is installed on one side of the subject's head and a second set of electrode assemblies 10′ is installed on the opposite side of the subject's head, and an AC voltage is applied between those two sets of electrode assemblies 10, an AC current will be capacitively coupled through the electrode assemblies 10′ into the subject's brain.

The layer of insulating material has a dielectric constant of at least 10. In some embodiments, the layer of insulating material 15 comprises a polymer layer having a thickness of less than 50 μm. In some embodiments, the layer of insulating material 15 comprises a ceramic material with a dielectric constant of at least 1000.

Returning to FIG. 1, one set of electrode assemblies 10 is installed on a first side (e.g. the left side) of the target region in the subject's brain through a respective first plurality of holes through the subject's skull, and a second set of electrode assemblies 10 is installed on the opposite side (e.g., the right side) of the target region through a respective second plurality of holes through the subject's skull. The installation of the electrode assemblies 10 may be implemented using the approaches described above in connection with FIG. 2, or any suitable alternative approach that positions the interior tip of each electrode assembly 10 against the subject's dura.

A port 40 is affixed to the subject's skull. This port 40 includes (a) at least one first terminal configured to make an electrical connection with each of the conductive wires 14 in the first set of electrode assemblies 10 on one side of the head (e.g., the left side), so that an electrical signal can be applied to each of those wires, and (b) at least one second terminal configured to make an electrical connection with each of the conductive wires 14 in the second set of electrode assemblies 10 on the opposite side of the head (e.g., the right side), so that an electrical signal can be applied to each of those wires. When there are more than two sets of electrode assemblies 10, the port 40 also includes at least one additional terminal configured to make an electrical connection with each of the conductive wires 14 in each of the additional sets of electrode assemblies 10, respectively, so that an electrical signal can be applied to each of those wires.

In alternative embodiments, instead of relying on a single port 40 to drive all of the sets of electrode assemblies (as depicted in FIG. 1), a dedicated port may be used to drive each of the sets of electrode assemblies 10, respectively. Thus, in these alternative embodiments, the number of dedicated ports will match the number of sets of electrode assemblies 10.

The AC voltage generator 30 operates at a frequency between 50 kHz and 1 MHz (e.g., between 100 kHz and 300 kHz). The output of the AC voltage generator 30 is connected to the port 40, and the port 40 routes the output of the AC voltage generator across opposing sets of electrode assemblies 10. For example, if the AC voltage generator 30 is generating a 200 kHz AC signal, the port 40 will route the 200 kHz output of the AC voltage generator 30 so that it appears across either (a) the left and right sets of electrode assemblies 10 or (b) the anterior and posterior sets of electrode assemblies 10.

When the AC voltage generator 30 applies its output signals across opposing sets of electrode assemblies 10, an alternating electric field will be induced (via either conductive coupling or capacitive coupling, as described above in connection with FIGS. 2 and 3, respectively) between the inner tips of the electrode assemblies 10 on one side of the target region and the inner tips of the electrode assemblies 10 on the other side of the target region. And notably, because the inner tips of each of the electrode assemblies 10 is located within the skull, that electric field will not have to traverse the subject's skull. As a result, voltages that are much lower than those used in the prior art Optune® system can be used to impose electric fields that are significantly stronger than the electric fields generated by the prior art Optune® system. And this improvement can increase the efficacy of treatment.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for delivering alternating electric fields to a target region in a subject's brain, the apparatus comprising:
a plurality of first electrode assemblies configured for positioning through a respective plurality of first holes in the subject's skull on a first side of the target region, wherein each of the plurality of first electrode assemblies has
a first shaft having a longitudinal axis, an inner end, and an outer end, wherein the first shaft is shaped and dimensioned to traverse a respective one of the first holes,
a first flange disposed at the outer end of the first shaft, wherein the first flange has a larger outer diameter than the first shaft and is shaped and dimensioned to prevent the first electrode assembly from passing through the respective first hole,
a first conductive electrode element disposed at the inner end of the first shaft, wherein the first electrode element has an inner face that is within 10° of perpendicular to the longitudinal axis of the first shaft, and wherein the inner face of the first electrode element has an area of at least 5 mm², and
a first conductive wire having a first portion that is positioned in electrical contact with the first electrode element and a second portion that is configured to run beneath the subject's scalp;
a plurality of second electrode assemblies configured for positioning through a respective plurality of second holes in the subject's skull on a second side of the target region, wherein each of the plurality of second electrode assemblies has
a second shaft having a longitudinal axis, an inner end, and an outer end, wherein the second shaft is shaped and dimensioned to traverse a respective one of the second holes,
a second flange disposed at the outer end of the second shaft, wherein the second flange has a larger outer diameter than the second shaft and is shaped and dimensioned to prevent the second electrode assembly from passing through the respective second hole,
a second conductive electrode element disposed at the inner end of the second shaft, wherein the second electrode element has an inner face that is within 10° of perpendicular to the longitudinal axis of the second shaft, and wherein the inner face of the second electrode element has an area of at least 5 mm², and
a second conductive wire having a first portion that is positioned in electrical contact with the second electrode element and a second portion that is configured to run beneath the subject's scalp; and
at least one port configured for affixation to the subject's skull, wherein the at least one port includes (a) at least one first terminal configured to make an electrical connection with each of the first conductive wires so that an electrical signal can be applied to each of the first conductive wires via the at least one port, and (b) at least one second terminal configured to make an electrical connection with each of the second conductive wires so that an electrical signal can be applied to each of the second conductive wires via the at least one port.

2. The apparatus of claim 1, wherein each of the plurality of first electrode assemblies has a first layer of insulating material disposed on the inner face of the first electrode element, wherein the first layer of insulating material is positioned to insulate the first electrode element from the subject's dura when the first shaft is positioned in the respective first hole, wherein the first layer of insulating material has a dielectric constant of at least 10, and
wherein each of the plurality of second electrode assemblies has a second layer of insulating material disposed on the inner face of the second electrode element, wherein the second layer of insulating material is positioned to insulate the second electrode element from the subject's dura when the second shaft is positioned in the respective second hole, wherein the second layer of insulating material has a dielectric constant of at least 10.

3. The apparatus of claim 2, wherein each of the first layers of insulating material and each of the second layers of insulating material comprises a polymer layer having a thickness of less than 50 μm.

4. The apparatus of claim 2, wherein each of the first layers of insulating material and each of the second layers of insulating material comprises a ceramic material having a dielectric constant of at least 1000.

5. The apparatus of claim 1, wherein each of the first electrode elements is positioned to contact the subject's dura when the respective first shaft is positioned in the respective first hole, and
wherein each of the second electrode elements is positioned to contact the subject's dura when the respective second shaft is positioned in the respective second hole.

6. The apparatus of claim 1, wherein the inner faces of each of the first electrode elements and each of the second electrode elements has an area of 5-20 mm².

7. A method for delivering alternating electric fields to a target region in a subject's brain, the method comprising:
positioning a plurality of first electrode assemblies through a respective plurality of first holes in the subject's skull on a first side of the target region, wherein each of the plurality of first electrode assemblies has
a first shaft having a longitudinal axis, an inner end, and an outer end, wherein the first shaft is shaped and dimensioned to traverse a respective one of the first holes,
a first flange disposed at the outer end of the first shaft, wherein the first flange has a larger outer diameter than the first shaft and is shaped and dimensioned to prevent the first electrode assembly from passing through the respective first hole,
a first conductive electrode element disposed at the inner end of the first shaft, wherein the first electrode element has an inner face that is within 10° of perpendicular to the longitudinal axis of the first shaft, and wherein the inner face of the first electrode element has an area of at least 5 mm², and
a first conductive wire having a first portion that is positioned in electrical contact with the first electrode element and a second portion that is configured to run beneath the subject's scalp;
positioning a plurality of second electrode assemblies through a respective plurality of second holes in the subject's skull on a second side of the target region, wherein each of the plurality of second electrode assemblies has
a second shaft having a longitudinal axis, an inner end, and an outer end, wherein the second shaft is shaped and dimensioned to traverse a respective one of the second holes,
a second flange disposed at the outer end of the second shaft, wherein the second flange has a larger outer diameter than the second shaft and is shaped and dimensioned to prevent the second electrode assembly from passing through the respective second hole,
a second conductive electrode element disposed at the inner end of the second shaft, wherein the second electrode element has an inner face that is within 10° of perpendicular to the longitudinal axis of the second shaft, and wherein the inner face of the second electrode element has an area of at least 5 mm², and
a second conductive wire having a first portion that is positioned in electrical contact with the second electrode element and a second portion that is configured to run beneath the subject's scalp; and
applying an alternating voltage between (a) the plurality of first electrode assemblies and (b) the plurality of second electrode assemblies, wherein the alternating voltage has a frequency between 50 kHz and 1 MHz.

8. The method of claim 7, wherein each of the plurality of first electrode assemblies has a first layer of insulating material disposed on the inner face of the first electrode element, wherein the first layer of insulating material is positioned to insulate the first electrode element from the subject's dura when the first shaft is positioned in the respective first hole, wherein the first layer of insulating material has a dielectric constant of at least 10, and
wherein each of the plurality of second electrode assemblies has a second layer of insulating material disposed on the inner face of the second electrode element, wherein the second layer of insulating material is positioned to insulate the second electrode element from the subject's dura when the second shaft is positioned in the respective second hole, wherein the second layer of insulating material has a dielectric constant of at least 10.

9. The method of claim 8, wherein each of the first layers of insulating material and each of the second layers of insulating material comprises a polymer layer having a thickness of less than 50 μm.

10. The method of claim 8, wherein each of the first layers of insulating material and each of the second layers of insulating material comprises a ceramic material having a dielectric constant of at least 1000.

11. The method of claim 7, wherein each of the first electrode elements is positioned to contact the subject's dura when the respective first shaft is positioned in the respective first hole, and
wherein each of the second electrode elements is positioned to contact the subject's dura when the respective second shaft is positioned in the respective second hole.

12. The method of claim 7, wherein the inner faces of each of the first electrode elements and each of the second electrode elements has an area of 5-20 mm².

13. The method of claim 7, wherein the alternating voltage has a frequency between 100 kHz and 300 kHz.

14. An apparatus for delivering alternating electric fields to a target region in a subject's brain, the apparatus comprising:
a shaft having a longitudinal axis, an upper end, and a lower end, wherein the shaft has a length of 4-10 mm and an outer diameter of 2-15 mm;
a flange disposed at the upper end of the shaft, wherein the flange has a diameter that is at least 2 mm larger than the outer diameter of the shaft;
a conductive electrode element disposed at the lower end of the shaft, wherein the electrode element has a lower face that is within 10° of perpendicular to the longitudinal axis of the shaft, and wherein the lower face of the electrode element has an area of at least 5 mm²; and
a layer of insulating material disposed on the lower face of the electrode element, wherein the layer of insulating material covers the lower face of the electrode element so as to prevent the lower face of the electrode element from coming into contact with tissue positioned below the apparatus, and wherein the layer of insulating material has a dielectric constant of at least 10.

15. The apparatus of claim 14, further comprising a metal wire having a first section that is disposed in electrical contact with the electrode element and runs through the shaft,
wherein the electrode element is made of metal.

16. The apparatus of claim 15, wherein the layer of insulating material comprises a polymer layer having a thickness of less than 50 μm.

17. The apparatus of claim 15, wherein the layer of insulating material comprises a ceramic material having a dielectric constant of at least 1000.

18. The apparatus of claim 14, wherein the lower face of the electrode element has an area of 5-20 mm².

19. The apparatus of claim 14, wherein the shaft has a cylindrical outer surface.

20. The apparatus of claim 14, wherein the lower face of the electrode element is within 2° of perpendicular to the longitudinal axis of the shaft.
